(19) United States
(12) Patent Application Publication
(10) Pub. No.: US 2008/0208100 A1
(43) Pub. Date: Aug. 28, 2008

(54) Method and apparatus for removal of cerumen

(76) Inventor: Ranan Wolff, Woodmere, NY (US)

Correspondence Address: Steven Horowitz;Counselor At Law, Suite 700, 295 Madison Avenue, New York, NY, 10017, US

(21) Appl. No.: 11/710,135

(22) Filed: Feb. 23, 2007

(51) Int. Cl. A61F 13/38

(52) U.S. Cl. 604/1

(57) ABSTRACT

A method of removing cerumen includes providing an applicator swab comprising an applicator stick having a swab tip mounted on a first end of the applicator stick, the swab tip including a medical grade pressure sensitive adhesive absorbed onto a top surface of said swab tip in a surface area of at least 25 square millimeters, the adhesive having a tack force sufficient to attach to and dislodge a large piece of cerumen from a person's external ear canal and hold it for at least three seconds. An end of the applicator swab is inserted into the external ear canal so that the top layer of the applicator tip contacts the cerumen. The applicator swab is then removed with the attached cerumen and the process is repeated as necessary after wiping off the cerumen.

### FIELD OF THE INVENTION

The field of this invention is methods and apparatuses for the removal of cerumen, and more particularly, such methods and apparatuses using applicator swabs to remove cerumen from the external ear canal of the human ear.

### BACKGROUND OF THE INVENTION AND DISCUSSION OF THE PRIOR ART

Cerumen, also known as ear wax, is normally produced by all individuals. It is produced by the ears to protect the skin of the ear from water. By keeping the skin of the ear dry, cerumen maintains the trumpet mechanism of the ear. It also protects against infection.

Cerumen is composed of dead skin cells and secretions from tiny ceruminous glands in the skin of the wall of the external ear canal (the outer part of the ear canal). Its purpose seems to be to block dust particles and water from entering into the ear. Cerumen is generally cleansed form the canal by washing of the body. Although cerumen is helpful, when it accumulates excessively it can adversely affect the person's hearing or the proper functioning of a hearing aid.

Individuals try to remove cerumen from the ear by using tissues or applicator swabs. This requires a certain amount of dexterity on the part of the person attempting to do the job. For example, one problem is that the external ear canal has a multitude of curved portions for acoustic and other reasons that make it easy for the cerumen to lodge securely in the ear and harder for it to be dislodged and removed. Prior to removal, the cerumen has to be first dislodged from the curved nooks in which it is lodged. It is usually unsanitary or awkward to try to use one's fingers to remove the cerumen and it is not easy to find something suitable to insert into the ear that can be used to grasp the cerumen. As a result, the cerumen, or at least part of the cerumen, has to be set into motion and then swept out on the applicator swab or tissue, preferably in one motion. This is not easy, can be frustrating and can waste time. Even if some comes out on the swab, it is not clear whether other parts of the cerumen remain lodged in the external ear canal.

An additional problem is that an applicator swab suitable for use with certain individuals may be unsuitable for use with other individuals. Individuals vary with respect to the quantity and stickiness of their cerumen. This variation correlates to some extent with ethnicity and age. Different ethnicities have dry or sticky cerumena and different ages may have different amounts of cerumen. Furthermore, the behavior of children differs from the behavior of adults. For example, when trying to remove cerumen from children and young adults, they may move suddenly. Consequently, there is a need for a method and apparatus for cerumen removal that can be tailored to people of different groups, such as different ages and ethnicities.

Another important problem in removing cerumen with the most commonly used applicator swabs is that these swabs, for example those made of cotton or similar materials, have next to no tackiness. As a result, the user tends to apply significant force against the cerumen, and hence against the walls of the external ear canal itself, in order to try to get the cerumen to attach itself to the swab. This kind of force is unhealthy and, if repeated, could lead to medical problems since it actually pushes the cerumen further into the ear canal. When the cerumen is pushed beyond the visible wall of the external ear canal, it can make removal of the cerumen by the user impossible and can require the user to undergo a medical procedure in a doctor's office to have it removed. Tightly packing the cerumen harder and harder can also eventually reduce the user's hearing abilities and can leave it more tightly embedded in the walls of the external ear canal.

A further problem is that other debris besides ear wax may need to be removed from the ear, for example in persons suffering from eczema. When this debris is white, white applicator tips do not adequately inform the user whether the debris has successfully been removed.

What is needed is a simple and practical way to remove unwanted all kinds of unwanted debris, called cerumen, from the ear safely, effectively, easily and inexpensively. It is further important that the method be readily useable by layman without special equipment. Most important, it is critical that the method be usable effectively without a significant amount of dexterity.

The method and apparatus of the present invention achieves all of these objectives as well as other many objectives.

### SUMMARY OF THE PRESENT INVENTION

In one preferred embodiment, the present invention is a method and apparatus for removing ear wax which method and apparatus can be tailored to individuals of different ages and ethnicities. The method includes providing an applicator swab comprising an applicator stick having a swab tip mounted on a first end of the applicator stick, the swab tip including a medical grade pressure sensitive adhesive absorbed onto a top surface of said swab tip, typically in a surface area of at least approximately 25 square millimeters, the pressure sensitive adhesive having a tack force sufficient to attach to and dislodge a large piece of cerumen in an ear canal of a person and hold the cerumen (and in some embodiments at least one tenth of a gram of cerumen) for at least three seconds when the applicator swab is held in any direction, including approximately parallel to the walls of the external ear canal. The method also includes inserting the first end of the applicator swab into an external ear canal of a person so that the top layer of the swab tip makes contact with the cerumen, and removing the applicator swab with the cerumen attached thereto.

### IMPORTANT OBJECTS AND ADVANTAGES

The following important objects and advantages of the present invention are:

(1) to provide a method and apparatus to remove cerumen from the external ear canal of a person;

(2) to provide such a method and apparatus that is safe for the skin by a variety of medically recognized safety standards, including in certain embodiments, internationally recognized safety standards;

(3) to provide such a method and apparatus that employs a pressure sensitive adhesive that is medical grade;

(4) to provide such a method and apparatus wherein the medical grade pressure sensitive adhesive is in certain embodiments a hot-melt acrylic that is first heated before applying it onto the tip of the applicator swab;

(6) to provide such a method and apparatus wherein the medical grade pressure sensitive adhesive is in certain embodiments a liquid having a viscosity of at least 300 cpi;

(7) to provide such a method and apparatus wherein the pressure sensitive adhesive is applied to the tip of the applicator swab by pouring, squeezing the liquid out or by dipping the tip into the liquid or by any other well known means

(9) to provide such a method and apparatus that is effective in the removal of cerumen from the external ear of humans;

(10) to provide such a method and apparatus that does not require use of expensive equipment;

(11) to provide such a method and apparatus that does not require significant manual dexterity;

(12) to provide such a method and apparatus that does not require manipulating the swab tip in various directions to dislodge the piece of cerumen;

(13) to provide such a method and apparatus that allows the width of the swab tip to be increased since manipulating the angle of the swab tip is no longer necessary;

(14) to provide such a method and apparatus wherein the swab tip has a width that is almost as thick as the width of the external ear canal of a user;

(15) to provide such a method and apparatus that can successfully remove large amounts of cerumen;

(16) to provide such a method and apparatus that is effective for people of a variety of ages including children adults and the elderly

(17) to provide such a method and apparatus that can be tailored to individuals of different ages;

(18) to provide such a method and apparatus that can be tailored to individuals of different races and/or ethnicities, such as Asians, native Americans, Caucasians, Afro-Americans;

(19) to provide such a method and apparatus that is easy to use;

(20) to provide such a method and apparatus that can make use of inexpensive and disposable applicators;

(21) to provide such a method and apparatus that can be used with available cotton applicator swabs already on the market by the addition of an appropriate adhesive;

(22) to provide such a method and apparatus wherein the adhesive can be applied to the applicator swab merely by spraying it on or dipping the swab tip in the adhesive;

(23) to provide such a method and apparatus wherein the pressure sensitive adhesive occupies the surface of the swab tip;

(24) to provide such a method and apparatus wherein the pressure sensitive adhesive occupies at least approximately 5 to approximately 15 square millimeters of surface area in certain embodiments;

(25) to provide such a method and apparatus wherein the pressure sensitive adhesive can be applied to both applicator tips on each side of an applicator swab;

(26) to provide such a method and apparatus wherein in certain embodiments each swab tip of an applicator swab has a pressure sensitive adhesive that is targeted and tailored to a different age ethnicity or other grouping of users than the adhesive on the other swab tip is tailored and targeted to;

(27) to provide such a method and apparatus wherein in certain alternative embodiments the pressure sensitive adhesive is on one swab tip of the applicator swab and the other swab tip has a dissolving agent, such as an oil, placed or embedded on or in the other swab tip;

(28) to provide a method and apparatus of the present invention as described in the above Objects and Advantages which is designed specifically to remove the debris caused in the ear canals of individuals suffering of eczema in the ear;

(29) to provide such a method and apparatus that is suitable for the removal of any debris in the ear of a human;

(30) to provide an applicator swab whose colored tip allows easy determination of whether cerumen pieces have successfully been attached to the tip;

(31) to provide a method and apparatus of using an applicator tip to remove cerumen from the ear that utilizes a medical grade adhesive that is tacky enough to do the job effectively;

(32) to provide a method and apparatus of using an applicator tip to remove cerumen from the ear that utilizes a medical grade adhesive that is tacky enough to do the job safely by avoiding the need to push the cerumen too hard; and

(33) to provide such a method and apparatus that is specifically designed to allow efficient removal of cerumen from the external ear canal of a person.

### BRIEF DESCRIPTION OF THE DRAWINGS

FIG. 1 is a sectional view of the swab used in the method and apparatus of the present invention wherein the applicator stick is made of paper;

FIG. 2 is a sectional view of one end of the swab used in the method and apparatus of the present invention wherein the applicator stick is made of wood;

FIG. 3 is a cross-sectional view of two ends of the swab used in the method and apparatus of the present invention, wherein each end has the adhesive embedded therein;

FIG. 4 is a sectional view of an alternative embodiment of a swab used in an alternative embodiment of the present invention;

FIG. 5 is a partial perspective view showing the apparatus of the present invention being used to remove cerumen as per the method of the present invention; and

FIG. 6 is a sectional view showing a further preferred embodiment of the swab of the present invention having a thicker diameter.

### DETAILED DESCRIPTION OF THE PREFERRED EMBODIMENT

“Cerumen”, which is the technical term for ear wax, is composed of dead skin cells and secretions from tiny ceruminous glands in the skin of the wall of the external ear canal (the outer part of the ear canal). Cerumen is sticky in texture in some people and it is dry in texture in some people. Cerumen is slightly acidic and is soluble in water

For the purposes of this patent application, such as in the claims, objectives and detailed description, the term “cerumen” shall be understood to be a broad term that includes but is not limited to ear wax. It also includes other debris in the ear such as dead skin cells alone or secretions from glands in the lining of the ear alone. Furthermore, it specifically also includes the debris in the ear that arises from individuals who have eczema in the ear. In general, although the present invention refers to the external ear canal of humans, the external ear canals of other species can benefit from the method and apparatus of the present invention.

The method and apparatus of the present invention will now be illustrated by reference to the accompanying drawings. The swab of the present invention has been assigned reference numeral 10. Other elements have been assigned the reference numerals referred to below.

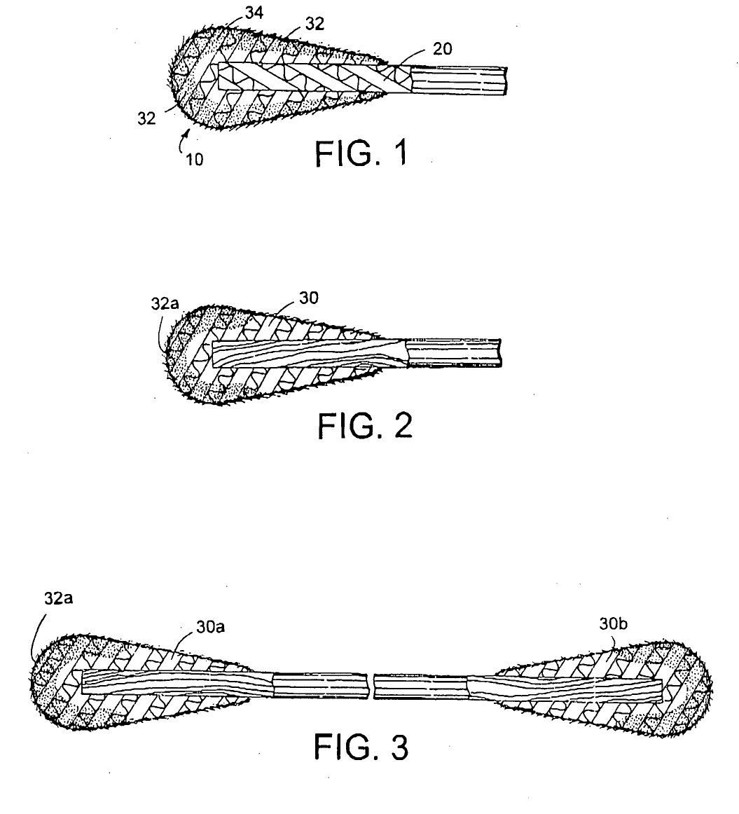

As seen from FIGS. 1-5, the apparatus of the present invention is an applicator swab 10, comprising an applicator stick 20 having a swab tip 30a mounted on at least a first end of the applicator stick. In one preferred embodiment, the applicator stock 20 also has a second swab tip 30b mounted on a second end of the applicator stick 20. At least one of the swab tips, 30a, 30b, and in one preferred embodiment, both swab tips 30a, 30b has a non-toxic medical grade pressure sensitive adhesive 32 absorbed onto a top layer 34 of said swab tip preferably over the entire top layer 34 of the tip 30a, 30b.

As discussed further below, the pressure sensitive adhesive has a tack force sufficient to attach to and dislodge even a large piece of cerumen in an ear canal of a person and hold the cerumen stuck in the ear for at least three seconds whether the applicator swab is held in any direction, including approximately parallel to the walls of the external ear canal. “Dislodge” in this context means dislodge the cerumen onto the swab tip 30. In a preferred embodiment, the pressure sensitive adhesive has a tack force sufficient to attach to cerumen in an ear canal and hold the cerumen for longer than a three seconds, for example at least ten seconds. In a further preferred embodiment, it can hold the cerumen for at least a minute. While a piece of cerumen may weigh only in the order of magnitude of one milligram, adhesive 32 should have a tack force that can lift something significantly heavier so as to be able to dislodge the cerumen. The two medical grade pressure sensitive adhesives discussed below can do this.

As discussed below, the pressure sensitive adhesive can be applied by dipping the swab tip 30a or 30b in the pressure sensitive adhesive or by pouring the pressure sensitive adhesive onto tip 30a or 30b. In certain alternative embodiments, pressure sensitive adhesive 32 may also be applied in any other manner known to those skilled in the art, including in theory spraying the adhesive.

The application of the pressure sensitive adhesive onto swab tip 30a or 30b should be such that enough pressure sensitive adhesive 32 permeates the top layer 34 of swab tip 30a or 30b. In certain embodiments, the pressure sensitive adhesive would also be absorbed into and embedded into a top layer 34 of the swab tip 30a or 30b. This is best seen from FIGS. 1-4 which depict adhesive 32 occupying a top layer 34 of swab tips 30a, 30b, said top layer 34 having a certain depth below the top layer 34 of the swab tips 30a, 30b.

Preferably, swab tip 30 is made of a soft material to avoid damaging the ear. Typically, swab tip 30 is made of a non-woven fibrous material that is absorbent. A common example of this material is cotton. Any natural or synthetic material that is suitable for mounting onto an applicator stick, that is safe (e.g. it would not abrasively damage the walls of the ear canal and would not leave harmful residue, is not toxic etc.) and that is absorbent is acceptable. In a preferred embodiment, the pressure sensitive adhesive 32 is embedded in a top layer 34 or portion of the swab tip 30.

In a further embodiment, the swab tip 30 is colored so that it contrasts with certain debris in the ear that is white. In particular sufferers of eczema have debris in the ear that is white like dandruff and would benefit from a swab tip 30 that can easily show whether the user has successfully removed the debris or not. The color of the cotton or other fibrous or other material of the swab tip 30 may be made by any means known to those skilled in the art, including dyeing.

Preferably, the adhesive 32 is absorbed into the depth of the cotton or other material of the swab tip 30 substantially uniformly. It is noted that when adhesive 32 is absorbed into the swab tip's top layer 34, adhesive 32 must still remain on the top layer 34 of swab tips 30a, 30b. This allows adhesive 32 to easily and quickly contact the cerumen when applicator stick 20 is inserted into the external ear canal of a person's ear and applied against the walls of the ear canal.

Recall that the top layer of the tip 30 wherein the embedded adhesive 32 is located is called the top layer 34. The uppermost top surface 32a of the adhesive 32 is located at the “uppermost” portion of top layer 34 of the swab tip 30. Here “uppermost” portion refers to the portion abutting the ambient. Uppermost top surface 32a must also have a minimum surface area in order to be effective in attaching to and holding cerumen. This is due to two factors, the first of which is that tack force is proportional to its surface area. The second is that the tip 30 will be most effective if it can grab and dislodge the most debris from the ear in the shortest amount of time and in this regard the larger the area in which attachment can occur the more work can be done.

As seen from FIGS. 1-5, in one preferred embodiment top layer 34 is a curved surface that includes either all of or at least a portion of the substantially hemispheric surface at the very top of swab tip 30 and all or portions of the substantially straight side of inverted cone 3. In one alternative version of the preferred embodiment, where tip 30 has a hemispheric top, top surface 32a of top layer 32 may not have to extend beyond that shown in FIGS. 1-5 since any further lengthening of top surface 32a downward would be of diminishing value given that the outer surface of the lower portion of the tip 30, situated after the tip 30 narrows, would be less likely to come into contact with the walls of the external ear. Moreover, in certain alternative embodiments, the band of thickness of the adhesive 32 would not extend across the full surface area of the swab tip to minimize smearing adhesive on the side walls of the ear when inserting the swab tip 30 into the ear to remove cerumen. This would be in cases where adhesive 32 is of an inferior kind and does “run” and leave residue, hence it is an alternative embodiment.

Based on crude estimates, if the swab tip 30 in cone-shaped and has a width at its widest point of d millimeters, then in order for the surface area on top layer 34 to be effective for the method and apparatus of the present invention, top layer 34 should have a surface area covering at least the hemispheric portion of the cone, which is equal approximately to

π/2 times d².

This is based on the surface of a hemisphere being equal to 2πr². Accordingly, if the swab 30 has a width, d, at its widest point, equal to approximately 6.5 millimeters (the approximate width of swabs sold under the brand name QTIP®, then if the adhesive 32 were embedded in the entire hemispheric portion of the cone but no more, the uppermost top surface 32a would equal approximately 66 and one third square millimeters. On the other hand, if the swab 30 has a width, d, at its widest point, equal to approximately 4 millimeters, the smallest imaginable width of a swab that would be at least minimally effective, then if the adhesive 32 were embedded in the entire hemispheric portion of the cone but no more, the uppermost top surface 32a would equal approximately 25 square millimeters.

If one assumes that to be practical and effective at all the uppermost top surface 32a must cover at least half of that hemisphere, then approximately 30 square millimeters is the minimum surface area for top surface 32a for a swab tip 30 that is 6.5 millimeters wide. In a preferred embodiment, however, the top surface 32a of the top layer 32 has a surface area that at least covers the entire hemispheric portion of the cone. Ideally, top surface 32a covers the entire top surface of the swab tip 30 adjacent the ambient air.

With a proper adhesive 32, there is not a significant concern that the surface area of top surface 32a should not cover all of the surface area of swab tip 30 to avoid causing adhesive on the top surface 32a to adhere to the side walls of the external ear canal after use of the apparatus and method of the present invention because a proper version of the adhesive 32 does not “run”. That is, it does not tend to leave residue when applied to a surface. Accordingly, in the preferred embodiment, top surface 32a of top layer 32 does extend across the entire surface area of the swab tip 30. This allows attaching to cerumen that is situated at any point along the side walls of the external ear canal.

In a further preferred embodiment, the adhesive 32 absorbed in the swab tip 30 need not necessarily reach the top of the applicator stick 20. Rather, adhesive 32 is preferably absorbed into a band of thickness, possibly a substantially uniform thickness, in a top layer of the swab tip.

In general, although FIG. 2 depicts the band of adhesive as having a particular thickness and as extending a particular amount down the sides of the swab tip 30, it should be understood that the present invention certainly contemplates other variations of both thickness and length of the band of adhesive 32.

Furthermore, in a further preferred embodiment, the applicator swab would not be shaped as shown in FIGS. 1-5 of the drawings herein but rather would be thicker as shown in FIG. 6. It may also be more cylindrical and less bulb-like as shown in FIG. 6 although not is not essential. It is believed that the reason applicator swabs for the ear are typically in the shape of FIGS. 1-5 is that ear wax is difficult to remove and in the course of trying to get the ear wax to stick to the swab the ear wax can easily fall off the cotton swab. This is true for example for cotton swabs sold under the name QTIP®, since they have limited tackiness. Accordingly, such a swab (without adhesive) requires dexterous manipulation and inclining of the applicator tip within the ear canal. In order to have room to do this the swab itself typically has a narrower diameter than the diameter of the ear canal and is shaped so that the swab tapers off and becomes narrower.

In the case of the present invention, however, where the dexterity and manipulation of the angle of the swab is no longer necessary due to the tackiness of the swab tip of the present invention, the shape and thickness of the swab tip 30 can be different. First, the width can be increased so that its widest diameter can be up to even equal to or slightly less than the diameter of the external ear canal. It should be understood that even the bulb-shaped swabs shown in FIG. 1-5 are typically between 6.5 millimeters and 10 millimeters at their widest point. This is contrast to swabs sold under the brand name QTIP®, which are approximately 6.5 millimeters wide at their widest point.

In addition, in a further preferred embodiment shown in FIG. 6, swab tip 30 of the apparatus of the present invention designed for an ear of an adult ear can be shaped substantially cylindrically although preferably with rounded corners. The width of the swab tip of FIG. 6 can vary but typically it would be approximately 10 millimeters, at least for adults of certain sizes. It is believed that at least some clearance between the swab and the walls of the external ear canal should be maintained to maximize ease of entry and exit to and from the ear canal. It is noted that the rounded corners of FIG. 6 are somewhat exaggerated and the embodiment shown in FIG. 6 more typically would have somewhat less rounded corners.

In a preferred embodiment, if the medical grade pressure sensitive adhesive 32 is a liquid it would preferably have a viscosity of at least 300 cpi. If the pressure sensitive adhesive 32 is hot-melt acrylic then its viscosity should be in the approximate range of 5,800 to 16,000 cpi measured at 350 degrees Fahrenheit.

In one preferred embodiment, pressure sensitive adhesive 32 is a hot-melt acrylic that is heated to a liquid state and then applied. It dries after application to the swab. Based on experimentation, a hot-melt acrylic pressure sensitive adhesive that has the requisite physical characteristics and that is medically safe can be found. For example, a hot-melt acrylic pressure sensitive adhesive 32 having product number HM-1003 is made by The Glue Factory, a company affiliated with or a division of Ellsworth Adhesives of 3430 W. Highview Drive, Appleton, Wis. (920-731-9016). After being melted from a solid, this hot-melt acrylic product has a Brookdale viscosity of 10,000 to 16,000 cps measured at 350 degrees Fahrenheit, which is believed to make it a little more viscous than room temperature molasses or chocolate syrup. It has a very low glass transition temperature (Tg) of −30, which is a measure of the softness of the polymer and it has a softening temperature of 105 degrees Centrigrade. Furthermore, this pressure sensitive adhesive 32 is has been tested and shown to meet U.S. Food & Drug Administration (“FDA”) criteria and U.S. Pharmacopeia (“USP”) criteria affecting safety to humans. It is listed as a “1501009 USP 6 class permanent” pressure sensitive adhesive. USP is the official public standards-setting authority for all prescription and over-the-counter medicines, dietary supplements, and other healthcare products manufactured and sold in the United States.

The pressure sensitive adhesive HM-1003 by The Glue Factory has been tested and has a tack force sufficient to attach to any piece of cerumen, dislodge it from the ear onto a surface having this adhesive and then hold it at least long enough to remove it from the ear. Furthermore, it unquestionably picks up significantly more cerumen than can be removed with a plain applicator swab lacking the adhesive used in the present invention.

Furthermore, the pressure sensitive adhesive HM-1003, based on experimentation, when embedded onto the top surface of an applicator tip of an applicator swab, has a tack force sufficiently strong to dislodge ear wax from the ear and hold the ear wax for significantly longer than a few seconds—more than enough time to remove it and start again. This is despite the fact that the cerumen itself is somewhat sticky and may be attached with a certain force to the walls of the external ear canal. This is also despite the fact that the cerumen may be embedded in a corner of the external ear canal or alternatively may be embedded in a crevice in the wall of the external ear canal. The HM-1003 pressure sensitive adhesive is able to dislodge, pick up and easily hold even a large piece of cerumen from the ear canal onto a swab tip 30 as part of a process of removing a whole series of individual pieces of cerumen from the ear. Furthermore, HM-1003 is viscous enough that it does not saturate the cotton or other fibrous material on the swab tip 30 so deeply. Hence, it allows only the quantity of adhesive needed to accomplish the task to be used and saves on the cost of manufacturing the product used in the present invention.

HM-1003 also is tacky enough to remove numerous pieces of ear wax or other debris from the ear within a short amount of time.

The Glue Factory also makes and sells other hot-melt acrylic pressure sensitive adhesives that would in all likelihood be suitable for the present invention. For example, HM-1002 having a viscosity of 15,000-16,000 cpi (measured at 350 degrees Fahrenheit) and a softening point of 150 degrees Centigrade and HM-1004 having a viscosity of 5,800 to 6,000 psi (measured at 350 degrees Fehrenheit) and a softening point of 95 degrees Centigrade are two other hot-melt acrylic pressure sensitive adhesive made by same company that makes HM-1003. However, as of the filing of this application, only HM-1003 has specifically been tested for safety and has been established to meet FDA and U.S. Pharmacopeia criteria.

In a further preferred embodiment, the pressure sensitive adhesive 32 is not a hot-melt acrylic but comes as a liquid and dries after application to the swab. For example, a product called A-4034 is a white water-based adhesive having a viscosity of 500-1000 cps has excellent tack and provides excellent adhesion to cloth. The A-4034 pressure sensitive adhesive, based on Applicant's experimentation, when embedded onto the top surface of an applicator tip of an applicator swab, is strong enough to dislodge even a large piece of ear wax or cerumen from the ear canal and hold the ear wax for significantly longer than several seconds—more than enough time to remove it and retain its strength to start again and thereby remove a whole series of individual pieces of cerumen from the ear. It is estimated that A-4034 could in fact hold cerumen weighing more than ten times that of a large piece of cerumen, based on its performance in experiments and its peel strength. In contrast to HM-1003, A-4034 is a less viscous liquid and saturates a greater depth of the cotton or other fibrous material on swab tip 30 when applied. This has the disadvantage that it uses more adhesive to accomplish the task to be used and costs more.

A-4034, which is manufactured by Ellsworth Adhesives of Appleton, Wis. (920-731-9016) has been tested (not by Applicant) for its potential to produce skin irritation and skin allergy in a controlled study with human volunteers. A-4034 was applied to the skin of 214 human volunteers in intervals of 24 or 96 hours for a period of three weeks. Irritation to the application site was recorded throughout. After a rest period of one week, volunteers were challenged with test material at a naïve site and responses recorded. Regarding irritation, it was concluded that the test material would be well tolerated by the majority of users. A small number of volunteers (25) had minimal erythema (redness). The hazard of sensitization is exceedingly small even under occlusive conditions based on the results of this study.

In addition A-4034 has the following physical properties.

| | |
|---|---|
| Type | Water-based Adhesive |
| Appearance | White |
| Solids | 52–56% |
| pH | 4.5–6.5 |
| Viscosity | 500–1000 cps @ 2/20/77° F. |
| 180° Peel (lbs/in width) | |
| 20 minute bond time | 1.7 |
| 24 hrs bond time | 1.7 (Transfer) |
| Shear Resistance (hrs) | |
| (1 lb/0.25 sq. in.) | 0.2 |
| Polyken Tack (grams) | 375 |
| Density | Avg. 8.5 lbs/gallon |
| Storage Stability | Store at 70–90° F. |
| Shelf Life | 3 months |
| Protect From Freezing | Yes |
| Mechanical Stability | Outstanding |
| ETO and Gamma Sterilizable | |

The components of A-4034 are as follows:

| Components | CAS No. | Concentration % |
|---|---|---|
| Acrylic multipolymer resin | 26634-78-6 | 48–53 |
| Water | 7732-18-5 | 42–47 |
| Ammonium salt of sulfated | 9051-57-4 | 1–5 |
| Nonylphenoxy poly (ethyleneoxy) | | |
| ethanol | | |
| 2-ethylhexyl acrylate | 103-1107 | <0.2 |
| Vinyl acetate | 108-05-4 | <0.06 |

All components of A-4034 conform to C.F.R. Section 175.105 relating to adhesives, C.F.R. Section 176.180 Components of paper & paperboard in contact with dry food and C.F.R. Section 176.170 relating to components of paper and paperboard in contact with aqueous and fatty foods (subject to the extraction limitations thereof).

Thus, the pressure sensitive adhesive used in the method and apparatus of the present invention is non-toxic for cosmetic use of human skin. Preferably, it should not only be non-toxic but it should be capable of being topically applied to the skin of a human being without irritation, disease or other adverse health side effect. “Medical grade” adhesive is used herein to mean an adhesive that besides being non-toxic to humans (i) meets criteria of the U.S. FDA or equivalent international body either for use in connection with adhesives and substances that come into contact with food or for use in connection with application to human skin or (ii) has been successfully subjected to a controlled scientific study of humans.

In a preferred embodiment, it should be considered by the Food & Drug Administration and the equivalent international bodies to be safe enough for topical application to facial skin. It should also preferably be considered safe enough for application to the external ear canal by the Food & Drug Administration for use in the U.S. and by the equivalent international bodies that govern use in their respective foreign countries. In a further preferred embodiment, it should not even cause minor irritation or even cause skin sensitivity. Ideally, it should also be safe enough that if some it is left or remains on the skin, no adverse reaction would occur.

Since safety guidelines of cosmetics and medical devices may vary as between adults and young children, for example children under the age of three, a particular version of the method and apparatus of the present invention may be applicable only to individuals above a certain age, for example the age of three years.

In certain preferred embodiments, the safety of the pressure sensitive adhesive complies with recognized guidelines for safety set forth in certain trade publications. For example, it complies with guidelines set forth in the Cosmetic Ingredient Review's (CIR) 2004 CIR Compendium, which contains concise information on nearly 1,200 safety assessments of individual cosmetic ingredients and is relied on by dermatologists, chemists, toxicologists, and industry and consumer safety groups. The CIR Compendium is a compilation of information taken from CIR's safety assessments which are based on the CIR's independent review of cosmetics.

Accordingly, in a preferred embodiment, the pressure sensitive adhesive is water-based. In certain preferred embodiments, the pressure sensitive adhesive employs a technology called microsphere technology. This helps to make the tack force of the pressure sensitive adhesive more stable over time. This means that adhesive 32 would be formed using microspheres, which are tiny particles measuring between 10 and 250 microns in diameter. It is known by those skilled in the art of making adhesives that microspheres are much larger than the tiny emulsion particles found in conventional adhesives. According to a company called Advanced Polymers International, located in Syracuse, N.Y., the size difference has a dramatic impact on adhesive tack. By forming a discontinuous film, microsphere adhesives limit physical contact, resulting in low peel, removability and stable tack over time. According to Advanced Polymers International emulsion adhesives, in contrast, use smaller particles which coalesce into a continuous film and do not allow for easy removal or repositioning.

It should be understood, however, that the present invention fully contemplates use of non-toxic medical grade pressure sensitive adhesives formed by emulsion or other techniques other than microsphere technology. At a minimum, the pressure sensitive adhesive 32 of the present invention need only have a tack force that is strong and stable enough to keep the cerumen on the swab tip 30 for a few seconds, or long enough to remove it from the external ear canal.

In one preferred embodiment, the pressure sensitive adhesive 32 is water-based to maximize its non-toxicity. This also has the advantage of making it less expensive. Furthermore, the pressure sensitive adhesive 32, in one preferred embodiment, is limited to non-toxic pressure sensitive adhesive that are not gel based. It is believed that a gel based pressure sensitive adhesive would have certain disadvantages for the method and apparatus of the present invention. For example, greater difficulty in uniformly penetrating and being easily absorbed into the top layer of the fibrous material on the swab tip. Furthermore, it is preferred that the pressure sensitive adhesive not extend above the top of the swab tip so that the shape of the swab tip is maintained for easy use. One also does not want a sticky gel to protrude unnecessarily out of the swab tip in case the user touches the swab tip accidentally. Such inadvertent contact would result in undesired smearing on the user but also would result in a deformation of the shape of the adhesive gel if the adhesive were a gel that protruded above the perimeter of the swab tip.

In certain preferred embodiments, the pressure sensitive adhesive should have a tack force sufficient to work effectively in accordance with the method of the present invention with pieces of cerumen having a wide range of weight and to be able to work for individuals of all ages. In other preferred embodiments, however, the method and apparatus of the present invention would make be tailored to specific age categories of the users, at least to some extent. For example, there would be an apparatus, and a method using said apparatus, that is suitable for adults, a different apparatus and method suitable for children above the age of two and a third kind of apparatus that is suitable for use on infants. There could also be an apparatus tailored to toddlers that differs from the apparatus suitable for older children.

In a further embodiment, the apparatus used in the method of the present invention can be tailored to individuals based on whether they tend to have dry or sticky cerumen in their ears. That is, different product lines can be sold for these different types of individuals. The basic method of the present invention would of course remain the same. For example, it is known that cerumen differs depending upon the racial background of an individual—for example cerumen is commonly sticky in whites and African Americans whereas cerumen is commonly dry in Asians and in native Americans.

The tack force of the pressure sensitive adhesive would vary depending upon whether the target cerumen was dry to sticky. This is because the tack force needed to pick up and hold a sticky substance is less than that needed to pick up and hold a dry substance. Accordingly, in a further preferred embodiment, the pressure sensitive adhesive used in the present invention would have a greater tack force when it is to be used on dry cerumen. Individuals who would use the apparatus and method of the present invention either know themselves whether their own cerumen is typically dry or sticky or else these individuals can be informed by marketing information on the packaging of a product sold to be used in the method or apparatus of the present invention that the product is for dry cerumen and that typically Asians and native Americans have dry cerumen.

Moreover, in general in all embodiments, the tack force should be sufficient to attach the cerumen to the swab tip and hold it long enough to remove the applicator swab from the ear. It has to be taken into account that the cerumen itself may in many cases be somewhat stuck to the walls of the ear. Thus in a preferred embodiment the tack force should be greater than the weight of the cerumen. Furthermore, although sticky cerumen is easier to cause to stick to the swab tip 30, it may also be harder to remove since it may be more stuck to the walls of the ear.

It is therefore very likely that an adhesive 32 that has the tack force necessary to dislodge the cerumen, especially a large piece of cerumen, will easily have the tack force necessary to hold it for several seconds or even much longer on the swab tip 30. This is because, as explained, the weight of the cerumen is probably much less than the force needed to dislodge the cerumen from the walls of the ear canal.

As used in the claims, the phrase “said pressure sensitive adhesive having a tack force sufficient to attach to and dislodge a large piece of cerumen in an ear canal of a person” means that the said pressure sensitive adhesive has a tack force sufficient to attach to and dislodge cerumen of any size commonly found in the ear canal of an adult person. As used in the claims the phrase “said pressure sensitive adhesive having a tack force sufficient to attach to and dislodge a large piece of cerumen in an ear canal of a child (or a person who is a child)” means that the said pressure sensitive adhesive has a tack force sufficient to attach to and dislodge cerumen of any size commonly found in the ear canal of a child.

This is determined empirically. One places the device 10 with the exposed pressure sensitive adhesive 32 adjacent to the cerumen with light pressure and then removes the swab. If cerumen appears on the swab tip 30 when the swab tip 30 is removed from the ear, or if it is detected that cerumen fell off the swab tip some time after removal of the swab tip from the ear, then the adhesive had the sufficient tack force to attach to and dislodge cerumen from the ear canal.

Furthermore, it has to be taken into account the fact that the ear is not an open area and hence there will be times when the swab tip will bump into the wall of the ear or other parts of the ear and that the cerumen may be subjected to a force other than its weight causing it to be dislodged from the swab tip. Hence the tack force should be sufficient to hold the cerumen securely enough to withstand these eventualities.

In certain preferred embodiments, the pressure sensitive adhesive has a tack force sufficient to remove and hold the cerumen for adults and children would thus be effective for a wide range of races since this level of tack force would be effective for cerumen that is dry or sticky.

In a preferred embodiment, it is believed that another requirement for the method and apparatus of the present invention is that the material holding the pressure sensitive adhesive, which is typically fibrous material such as cotton, which material is located on the tip 20a of the applicator stick 20, be sufficiently attached to applicator stick 20 of swab 10. In particular, the material should be attached to the applicator stick 20 with greater tack force than the tack force needed to dislodge the cerumen from the external ear canal. Otherwise, the material itself will be dislocated from the applicator stick 20 when trying to remove the cerumen and the cerumen will continue to be stuck to the external ear canal. The material would then be stuck in and clog the external ear canal, which is clearly undesirable. Thus, the tack force of the adhesive 32 should be less than the tack force holding the material (e.g. cotton) to the applicator stick 20.

The swab 10 can be a swab having a swab tip at both ends or having a swab tip 20 at only one end. Accordingly, pressure sensitive adhesive 32 can be embedded in a single swab tip 20 of the swab or it can be embedded in two swab tips off the swab, one at each end.

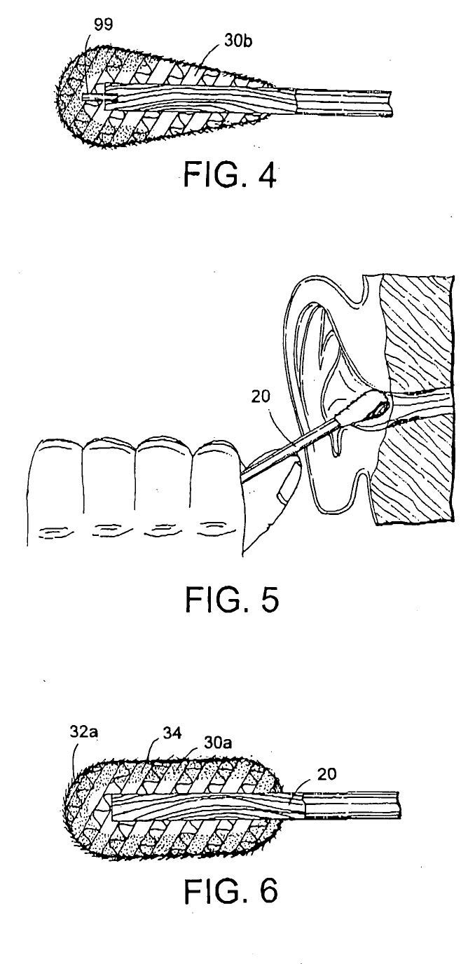

As best seen from FIG. 4, in a further alternative embodiment, the swab tip 30 has a first swab tip 30a with adhesive (not shown) and a second swab tip 30b at the second end having a tiny container 99 with a fluid dissolving agent in it. It is known that olive oil for example is useful in dissolving cerumen. Thus, the user can first apply the dissolver and then apply the end of the swab 10 having the adhesive on the swab tip for removal. This makes removal easier, particularly in cases where the cerumen is lodged tightly in the ear and is stuck to the walls of the ear.

### The Methods of the Present Invention

Based on the above, one embodiment of the method of the present invention is a method of removing a cerumen from the ear of a person, and comprises providing an applicator swab, the applicator swab comprising an applicator stick having a swab tip 30 mounted on a first end of the applicator stick, the swab tip including a medical grade pressure sensitive adhesive 32 absorbed onto a top surface of said swab tip in a minimum surface area of between approximately 5 square millimeters and approximately 10 square millimeters, said pressure sensitive adhesive having a tack force sufficient to attach to and dislodge a large piece of cerumen in an ear canal of a person and hold it (or in certain embodiments even hold at least one tenth of one gram of cerumen) for at least ten seconds when the applicator swab is held in any direction.

In another embodiment of the method of the present invention, the method includes the above step of providing the applicator swab but also includes the step(s) of inserting a first end of the applicator swab into an external canal of an outer ear of a person so that the top layer 34 of the swab tip attaches to a piece of the cerumen, and removing the applicator swab with the cerumen attached thereto.

In a further embodiment of the method of the present invention, the method comprises taking an applicator swab as described as per the previously listed version of the method and also includes the step(s) of inserting a first end of the applicator swab into an external canal of an outer ear of a person so that the top layer 34 of the swab tip attaches to a piece of the cerumen, and removing the applicator swab with the cerumen attached thereto.

In a still further embodiment of the method of the present invention, the method includes providing an applicator swab as described in the previous versions and also includes the step of instructing users of the applicator swab to insert the first end of the applicator swab into an external canal of an outer ear of a person to contact the cerumen.

In a still further embodiment of the method of the present invention, the method comprises (a) providing an applicator swab, the applicator swab comprising an applicator stick having a swab tip mounted on a first end of the applicator stick, and adding a medical grade pressure sensitive adhesive so that the pressure sensitive adhesive is absorbed onto a top surface of said swab tip in a minimum surface area of between approximately 5 square millimeters and approximately 10 square millimeters, said pressure sensitive adhesive having a tack force sufficient to attach to and dislodge a large piece of cerumen in an ear canal of a person and hold it (or in certain embodiments hold at least one tenth of one gram of cerumen) for at least ten seconds when the applicator swab is held approximately parallel to the walls of the external ear canal or in any other direction.

With respect to each of the above embodiments of the method of the present invention, further versions of each embodiment include having the pressure sensitive adhesive absorbed and embedded into a top layer 34 of the swab tip 30a, 30b. Furthermore, in a further version of each embodiment, in certain preferred embodiments, the top layer 34 is substantially uniform in width and/or depth.

Moreover, the step of providing the applicator swab with the swab tip that includes the pressure sensitive adhesive involves, in certain preferred embodiments, providing the applicator swab with the swab tip that includes a water-based non-gel pressure sensitive adhesive. In addition, the step of providing the applicator swab with the swab tip includes, in certain preferred embodiments, providing the applicator swab with a second swab tip on a second end of the applicator stick, the second swab tip made of cotton.

It is to be understood that while the method and apparatus of the present invention have been described and illustrated in detail, the above-described embodiments are simply illustrative of the principles of the invention. It is to be understood also that various other modifications and changes may be devised by those skilled in the art which will embody the principles of the invention and fall within the spirit and scope thereof. It is not desired to limit the invention to the exact construction and operation shown and described. The spirit and scope of this invention are limited only by the spirit and scope of the following claims.

What is claimed is:

1. An applicator swab for the removal of ear debris, comprising:
an applicator stick having a swab tip mounted on a first end of the applicator stick, the swab tip including a medical grade pressure sensitive adhesive absorbed onto a top surface of said swab tip, said pressure sensitive adhesive having a tack force sufficient to attach to and dislodge a large piece of cerumen in an ear canal of a person and hold said large piece of cerumen for at least three seconds regardless of a direction in which the applicator swab is held.

2. The applicator swab of claim 2, wherein the pressure sensitive adhesive has a minimum Brookdale viscosity of approximately 300 cpi.

3. The applicator swab of claim 2, wherein the pressure sensitive adhesive is absorbed onto a top surface of said swab tip in a surface area of at least 25 square millimeters.

4. The applicator swab of claim 1, wherein the swab tip is made of a fibrous non-woven absorbent material.

5. The applicator swab of claim 4, wherein the material is cotton.

6. The applicator swab of claim 3, wherein the top layer is substantially uniform in width.

7. The applicator swab of claim 1, wherein the applicator stick has a second swab tip mounted on a second end of the applicator stick.

8. The applicator swab of claim 1, wherein the pressure sensitive adhesive is a water-based non-gel pressure sensitive adhesive.

9. The applicator swab of claim 1, wherein the pressure sensitive adhesive is a hot-melt acrylic.

10. The applicator swab of claim 9, wherein the hot-melt acrylic has a high enough viscosity such that application of said adhesive on to the swab tip does not saturate all of the swab tip.

11. The applicator swab of claim 1, wherein the pressure sensitive adhesive is a hot-melt acrylic having a Brookdale viscosity of at least approximately 10,000 cps.

12. The applicator swab of claim 1, wherein the pressure sensitive adhesive has a peel strength of between approximately 1.5 and approximately 2.

13. The applicator swab of claim 1 wherein the applicator stick has a second swab tip mounted on a second end.

14. The applicator swab of claim 1, where the person is a child.

15. The applicator swab of claim 14, wherein the pressure sensitive adhesive has a minimum Brookdale viscosity of approximately 300 cpi.

16. The applicator swab of claim 14, wherein the pressure sensitive adhesive is a hot-melt acrylic.

17. The applicator swab of claim 16, wherein the hot-melt acrylic has a high enough viscosity such that application of said adhesive on to the swab tip does not saturate all of the swab tip.

18. The applicator swab of claim 14, wherein the pressure sensitive adhesive is a hot-melt acrylic having a Brookdale viscosity of at least approximately 10,000 cps.

19. A method of removing a cerumen from an ear canal of a person, comprising the steps of:
(a) providing an applicator swab, the applicator swab comprising an applicator stick having a swab tip mounted on a first end of the applicator stick, the swab tip including a medical grade pressure sensitive adhesive absorbed onto a top surface of said swab tip in a surface area of at least approximately 5 square millimeters, said pressure sensitive adhesive having a tack force sufficient to attach to and dislodge a large piece of cerumen in an ear canal of a person and hold said large piece of cerumen for at least three seconds regardless of a direction in which the applicator swab is held,
(b) inserting a first end of the applicator swab into an external canal of an outer ear of a person so that the top surface of the swab tip attaches to a piece of the cerumen,
(c) removing the applicator swab with the cerumen attached thereto.

20. The method of claim 19, where the person is a child.

21. The method of claim 19, wherein the pressure sensitive adhesive has a minimum Brookdale viscosity of approximately 300 cpi.

22. The method of claim 19, wherein the pressure sensitive adhesive is a hot-melt acrylic.

23. The method of claim 22, wherein the hot-melt acrylic has a high enough viscosity such that application of said adhesive on to the swab tip does not saturate all of the swab tip.

24. The method of claim 19, wherein the pressure sensitive adhesive is a hot-melt acrylic having a Brookdale viscosity of at least approximately 10,000 cps.

25. The method of claim 19, wherein the applicator stick has a second swab tip mounted on a second end, the method also including a step (d) of repeating steps (b) and (c) using the swab tip on the second end to remove additional cerumen.

26. The method of claim 19, wherein the pressure sensitive adhesive is absorbed into a top layer of the swab tip.

27. The method of claim 26, wherein the top layer is substantially uniform in width.

28. The method of claim 19, wherein providing the applicator swab with the swab tip that includes the pressure sensitive adhesive involves providing the applicator swab with the swab tip that includes a water-based non-gel pressure sensitive adhesive.

29. The method of claim 19, wherein providing the applicator swab with the swab tip that includes the pressure sensitive adhesive involves providing the applicator swab with the swab tip that includes a hot-melt acrylic medical grade pressure sensitive adhesive.

30. The method of claim 19, wherein providing the applicator swab with the swab tip includes providing the applicator swab with a second swab tip on a second end of the applicator stick, the second swab tip made of cotton.

31. A method of cleaning the ear, comprising:
(a) taking an applicator swab, the applicator swab comprising an applicator stick having a swab tip mounted on a first end of the applicator stick, the swab tip including a medical grade pressure sensitive adhesive absorbed onto a top surface of said swab tip in a surface area of at least approximately 5 square millimeters, said pressure sensitive adhesive having a tack force sufficient to attach to and dislodge a large piece of cerumen in an ear canal of a person and hold said large piece of cerumen for at least three seconds regardless of a direction in which the applicator swab is held,
(b) inserting the first end of the applicator swab into an external canal of an outer ear of a person so that the top surface of the swab tip makes contact with the cerumen, and
(c) removing the applicator swab with the cerumen attached thereto.

32. The method of claim 31, wherein the applicator stick has a second swab tip mounted on a second end, the method also including a step (d) of repeating steps (b) and (c) using the swab tip on the second end to remove additional cerumen.

33. The method of claim 31, wherein the pressure sensitive adhesive is absorbed into a top layer of the swab tip.

34. The method of claim 31, wherein the top layer is substantially uniform in width.

35. The method of claim 31, wherein taking the applicator swab with the swab tip that includes the pressure sensitive adhesive involves providing the applicator swab with the swab tip that includes a water-based non-gel pressure sensitive adhesive.

36. The method of claim 31, wherein taking the applicator swab with the swab tip includes taking the applicator swab with a second swab tip on a second end of the applicator stick, the second swab tip made of cotton.

37. A method, comprising:
(a) providing an applicator swab, the applicator swab comprising an applicator stick having a swab tip mounted on a first end of the applicator stick, the swab tip including a medical grade pressure sensitive adhesive absorbed onto a top surface of said swab tip in a minimum surface area of at least approximately 5 square millimeters, said pressure sensitive adhesive having a tack force sufficient to attach to and dislodge a large piece of cerumen in an ear canal of a person and hold said large piece of cerumen for at least three seconds regardless of a direction in which the applicator swab is held, and
(b) instructing users of the applicator swab to insert the first end of the applicator swab into an external canal of an outer ear of a person to contact the cerumen.

38. The method of claim 37, wherein the pressure sensitive adhesive is absorbed into a top layer of the swab tip.

39. The method of claim 37, wherein the top layer is substantially uniform in width.

40. The method of claim 37, wherein providing the applicator swab with the swab tip that includes the pressure sensitive adhesive involves providing the applicator swab with the swab tip that includes a water-based non-gel pressure sensitive adhesive.

41. The method of claim 37, wherein the applicator stick has a second swab tip mounted on a second end, the method also including a step (d) of repeating steps (b) and (c) using the swab tip on the second end to remove additional cerumen.

42. A method of making an improved applicator tip, comprising:
(a) providing an applicator swab, the applicator swab comprising an applicator stick having a swab tip mounted on a first end of the applicator stick,
(b) adding a medical grade pressure sensitive adhesive so that the pressure sensitive adhesive is absorbed onto a top surface of said swab tip in a surface area of at least approximately 5 square millimeters, said pressure sensitive adhesive having a tack force sufficient to attach to and dislodge a large piece of cerumen in an ear canal of a person and hold said large piece of cerumen for at least three seconds regardless of a direction in which the applicator swab is held.

43. The method of claim 42, wherein the pressure sensitive adhesive is absorbed into a top layer of the swab tip.

44. The method of claim 43, wherein the top layer is substantially uniform in width.

45. The method of claim 42, wherein providing the applicator swab with the swab tip that includes the pressure sensitive adhesive involves providing the applicator swab with the swab tip that includes a water-based non-gel pressure sensitive adhesive.

46. The method of claim 42, wherein the applicator stick has a second swab tip mounted on a second end, the method also including a step (d) of repeating steps (b) and (c) using the swab tip on the second end to remove additional cerumen.
